(19) United States
(12) Patent Application Publication
(10) Pub. No.: US 2014/0276245 A1
(43) Pub. Date: Sep. 18, 2014

(54) SLEEP EVALUATION DEVICE AND PROGRAM FOR SLEEP EVALUATION

(71) Applicants: OMRON HEALTHCARE Co., Ltd., Muko-shi, Kyoto (JP); OMRON Corporation, Shimogyo-ku, Kyoto-shi, Kyoto (JP)

(72) Inventors: Masakazu Tsutsumi, Kyoto (JP); Yoko Kanemitsu, Kyoto (JP); Yasuko Emori, Kyoto (JP); Masanori Hashizaki, Kyoto (JP); Naoki Tsuchiya, Kyoto (JP)

(21) Appl. No.: 14/354,184

(22) PCT Filed: Oct. 19, 2012
(86) PCT No.: PCT/JP2012/077093

§ 371 Date: Apr. 25, 2014

(30) Foreign Application Priority Data
Oct. 31, 2011 (JP) 2011-238571

(51) Int. Cl. A61B 5/00; A61B 5/11

(52) U.S. Cl. CPC A61B 5/4812; A61B 5/11; A61B 5/7278
USPC 600/595

(57) ABSTRACT

A body motion detection device detects a body motion of a subject. A result of detection is transmitted to a server where a determination is made as to a plurality of patterns of sleep (for example, “having difficulty falling asleep,” “awakening too early,” “awakening in the middle of night,” “shortage of sleep,” “sleep rhythm disturbance,” and “uninterrupted sleep”) based on the detection result.

### BACKGROUND OF THE INVENTION

1. Field of the Invention

The present invention relates to a sleep evaluation device and a program for sleep evaluation, and more particularly to a sleep evaluation device for non-invasively evaluating a sleep state of a subject and a program for sleep evaluation.

2. Description of the Related Art

Various techniques related to devices for evaluating sleep have been disclosed.

For example, Japanese Patent Laying-Open No. 2005-209143 discloses a technique in which a computer accepts answers from a user to questions as to whether sleep quality is good, whether a sleeping process is good, and actions after wakeup until wakeup, analyzes the input results to extract sleeping process factors and action factors that influence the sleep quality, and gives advice on how to improve sleep.

The technique disclosed in Japanese Patent Laying-Open No. 2005-209143 displays the analysis result on a basis of the contents based on the answers entered by the user. According to this technique, the analysis result can reflect the symptoms that the user is conscious of. A variety of techniques that are based on objective data have also been proposed.

For example, Japanese Patent Laying-Open No. 2007-319238 discloses a technique in which sleep evaluation items (total sleep duration, non-REM sleep, REM sleep, middle-of-the-night awakenings, and the like) of the measured sleep state are compared with mean values or standard deviations from normal population data, whereby sleep of a subject is represented in two or more levels, and sleep improvement advice is presented accordingly.

In presenting advice on improvement in sleep, it is always requested that a sleep tendency of a subject should be ascertained more accurately based on the acquired data and the like.

### SUMMARY OF THE INVENTION

In view of the aforementioned situation, preferred embodiments of the present invention provide a sleep evaluation device and a program for sleep evaluation that enables more accurate ascertainment of a sleep tendency of a subject.

A sleep evaluation device according to an aspect of a preferred embodiment of the present invention includes an acquisition unit configured to acquire a detection result as to movement of a body of a subject in bed, a first determination unit configured to determine a sleep state of the subject for each day, based on the detection result, and a second determination unit configured to determine a tendency of the sleep state of the subject, based on determination results for a plurality of days by the first determination unit.

The second determination unit determines a tendency of the sleep state of the subject, for a plurality of items. The plurality of items include items having different periods required for determination. The second determination unit gives a notice of the tendency of the sleep state of the subject, on the condition that a period required for determination for all the items of the plurality of items is expired after the first determination unit starts determination.

Preferably, the items for which the tendency is determined by the second determination unit include an item based on a proportion of a time during which the body is not moved in the detection result, to a sleep duration.

According to another preferred embodiment of the present invention, a non-transitory computer-readable medium includes a computer program for performing, when the computer program runs on a computer, a method of evaluating sleep of a subject. The computer program causes the computer to execute the steps of acquiring a detection result for movement of a body of a subject in bed that is detected by a body motion detection unit, determining a sleep state of the subject for each day, based on a detection result of the body motion detection unit, and determining a tendency of the sleep state of the subject, based on determination results of the sleep state for a plurality of days.

The step of determining a tendency of the sleep state of the subject determines a tendency of the sleep state of the subject, for a plurality of items. The plurality of items include items having different periods required for determination. The step of determining a tendency of the sleep state of the subject gives a notice of the tendency of the sleep state of the subject, on the condition that a period required for determination for all the items of the plurality of items is expired after the determination of a sleep state of the subject for each day is started.

According to various preferred embodiments of the present invention, a sleep state of a subject preferably is determined for each day, and a tendency of the sleep state of the subject is further determined based on the determination results for a plurality of days.

Accordingly, the tendency of the sleep state based on the sleep state for a plurality of days is provided to the subject, so that the sleep state of the subject is ascertained more accurately.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a diagram showing a specific example of a configuration of a sleep evaluation system according to a preferred embodiment of the present invention.

FIG. 2 is a schematic diagram depicting a side surface of a body motion detection device.

FIG. 3 is a schematic diagram showing an external appearance of the body motion detection device as viewed from the obliquely upper direction.

FIG. 4 is a block diagram showing a specific example of a hardware configuration of the body motion detection device.

FIG. 5 is a diagram illustrating a usage example of the body motion detection device.

FIG. 6 is a block diagram showing a specific example of a functional configuration to determine a sleep level in the body motion detection device.

FIG. 7 is a diagram showing a specific example of a sensor signal from a body motion sensor in the body motion detection device.

FIGS. 8A and 8B are diagrams showing a specific examples of a respiratory waveform and a body motion waveform separated from the waveform depicted in FIG. 7.

FIG. 9 is a diagram showing a specific example of determination results in the body motion detection device.

FIG. 10 is a diagram schematically showing an example of a hardware configuration of a server.

FIG. 11 is a diagram showing an example of a functional configuration of the server.

FIGS. 12A-12C are diagrams illustrating a mode of deciding a sleep level at predetermined intervals.

FIG. 13 is a diagram showing an example of the names of patterns determined in the server and the definition of criteria.

FIG. 14 is a diagram illustrating an example of a method of calculating standard deviations of the wake-up time.

FIG. 15 is a diagram illustrating contents of processing executed on a result for each day in the sleep evaluation system.

FIG. 16 is a diagram illustrating contents of processing for each day from the second day of measurement to the day previous to the last day (the thirteenth day in the present preferred embodiment).

FIG. 17 is a diagram illustrating contents of processing to determine a sleep tendency for a plurality of days (for a certain period of time) which are a period of time to provide a final determination result to a subject.

FIG. 18 is a diagram showing an example of a screen to display the result of determination of a sleep tendency.

### DETAILED DESCRIPTION OF THE PREFERRED EMBODIMENTS

Preferred embodiments of the present invention will be described below with reference to the figures. In the following description, the same elements and components are denoted with the same reference signs. Their names and functions are also the same.

FIG. 1 is a diagram showing a specific example of a configuration of a sleep evaluation system according to the present preferred embodiment.

Referring to FIG. 1, the sleep evaluation system according to the present preferred embodiment includes a body motion detection device 100, a server 500, and a user terminal 200, which are connected via a network such as a LAN (Local Area Network). The network may be either wired or wireless.

In the sleep evaluation system in the present preferred embodiment, body motion detection device 100 measures physical quantities related to body motions of a subject during sleep and transmits the measurement result to server 500. Server 500 processes the measurement result to calculate an index for a sleep tendency of the subject. User terminal 200 accesses server 500 to acquire data to display the index. Accordingly, the subject operates user terminal 200 to acquire information as to the state of his/her sleep.

Referring to FIG. 2 and FIG. 3, an external appearance of body motion detection device 100 will be described below. FIG. 2 is a schematic diagram depicting a side surface of body motion detection device 100. FIG. 3 is a schematic diagram showing an external appearance thereof as viewed from the obliquely upper direction.

Referring to FIG. 1 to FIG. 3, body motion detection device 100 has an external appearance in which a casing that preferably is a rectangular or substantially rectangular parallelepiped or elongated in shape with rounded corners, for example, is placed on a base. The external appearance of body motion detection device 100 is not limited thereto.

Operation button group 10 is disposed on a surface of the base. A display unit 20 is disposed on the surface of the casing placed on the base. The casing contains a sensor 30 and a control unit 40. Button group 10 includes a delete button 10A, a go-to-bed button 10B, a good night button 10C, a cancel button 10D, and a data processing button 10E. In the following description, the surface of the casing that is provided with display unit 20 is also called the front of body motion detection device 100.

Body motion detection device 100 includes a communication unit 50 configured for wired or wireless communication. Communication unit 50 is provided at the end of the casing that is opposite to the base. Body motion detection device 100 communicates with server 500 and user terminal 200 such as a mobile phone through communication unit 50.

FIG. 4 is a block diagram showing a specific example of a hardware configuration of body motion detection device 100.

Referring to FIG. 4, body motion detection device 100 includes control unit 40 that entirely controls operation of body motion detection device 100. Button group 10, sensor 30, display unit 20, and communication unit 50 all are connected to control unit 40.

Button group 10 outputs to control unit 40 an operation signal produced by being operated by the subject.

Sensor 30 includes a body motion sensor 31 configured to output a signal (hereinafter also called “sensor signal”) generated in body motion sensor 31 to control unit 40. Body motion sensor 31 preferably is implemented, for example, by a Doppler sensor. In the following description, it is assumed that body motion sensor 31 is a Doppler sensor. Another example of body motion sensor 31 is an ultrasonic sensor.

Body motion sensor 31 that is a Doppler sensor includes an output unit configured to output radio waves for measurement and a reception unit, though not shown. The reception unit receives radio waves reflected from a surface of the subject, of radio waves output from the output unit, and outputs a sensor signal in accordance with a change in frequency from the output radio waves.

In body motion detection device 100, a body motion of the subject may be detected by a camera instead of body motion sensor 31. In this case, body motion detection device 100 includes a camera instead of body motion sensor 31, and control unit 40 analyzes an image captured by the camera. A body motion is detected based on the result of the analysis.

Control unit 40 includes a CPU 41 configured and programmed to perform the entire control and a memory 42 configured to store a program executed in CPU 41.

Control unit 40 executes processing such as determination of a sleep state described later, by CPU 41 executing the computer program stored in memory 42 and executing operations using the input operation signal and sensor signal.

Memory 42 may be fixed to body motion detection device 100 or may be implemented by a detachable storage medium. Examples of the storage medium include a CD-ROM (Compact Disc-Read Only Memory), a DVD-ROM (Digital Versatile Disk-Read Only Memory), a USB (Universal Serial Bus) memory, a memory card, an FD (Flexible Disk), a hard disk, a magnetic tape, a cassette tape, an MO (Magnetic Optical Disc), an MD (Mini Disc), an IC (Integrated Circuit) card (excluding a memory card), an optical card, a mask ROM, an EPROM, an EEPROM (Electronically Erasable Programmable Read-Only Memory), and any other media storing programs in a nonvolatile manner.

Communication unit 50 is implemented, for example, by a LAN card. The mode of communication executed by communication unit 50 may be, for example, wireless communication such as infrared communication and communication using Bluetooth (Registered Trademark) that allows direct communication with user terminal 200, or may be communication with user terminal 200 through the Internet using the Internet connection function.

Communication unit 50 may additionally be configured to perform a server function of a wireless LAN (Local Area Network) to transmit display data as described later written in a markup language such as HTML (Hyper Text Markup Language), to user terminal 200 that has accessed through wireless LAN connection.

Body motion detection device 100 includes a timer 60. Timer 60 is connected to control unit 40. CPU 41 acquires time information from timer 60 and specifies the time, for example, the go-to-bed time as described later, for storage into memory 41.

FIG. 5 is a diagram illustrating a usage example of body motion detection device 100.

Referring to FIG. 5, body motion detection device 100 is installed in the vicinity (for example, at the bedside) of the subject during sleep, by way of example. Measurement operation is performed in this state, such that body motion sensor 31, which preferably is a Doppler sensor, outputs radio waves.

The radio waves output from body motion sensor 31 mainly arrive at the neighborhood of the chest and shoulders of the subject during sleep, and a change in frequency of waves reflected therefrom is output as a sensor signal to control unit 40. Control unit 40 detects a body motion, for example, movement of the chest and rolling over of the subject during sleep, and determines a sleep level based on the detection result.

FIG. 6 is a block diagram showing a specific example of a functional configuration to determine a sleep level in body motion detection device 100. The functional units shown in FIG. 6 are defined by CPU 41 being configured to execute the computer program stored in memory 42 and may at least be partially defined by a hardware configuration such as an electric circuit, for example.

Referring to FIG. 6, body motion detection device 100 includes an input unit 401 configured to accept input of a sensor signal from sensor 30, a sleep state measurement unit 402 configured to determine a sleep state in a unit period based on the sensor signal, a read unit 406 configured to read out display data from memory 42, a display control unit 407 configured to execute a process of allowing display unit 20 to display the read display data, and a communication control unit 408 configured to allow communication unit 50 to perform a process of transmission to user terminal 200.

Body motion detection device 100 also includes an input information processing unit 410 configured to process input information from a variety of buttons included in button group 10.

In the example shown in FIG. 6, input unit 401 directly receives a sensor signal from sensor 30. However, the sensor signal may be temporarily stored in a predetermined area of memory 42, and input unit 401 may read out the sensor signal therefrom when performing operation for display.

A method of determining a sleep state by sleep state measurement unit 402 will be described.

FIG. 7 is a diagram showing a specific example of a sensor signal from body motion sensor 31, which preferably is a Doppler sensor, for example. FIG. 7 shows temporal changes of voltage values related to the amount of phase shift between a carrier from the body motion sensor and a reflected wave from a surface of the subject.

Referring to FIG. 7, the waveform represented as the sensor signal is a composite wave including a waveform (hereinafter also referred to as respiratory waveform) representing a body motion (movement of the chest) associated with breathing of the subject and a waveform (hereinafter also referred to body motion waveform) representing body motions (movement of the body) other than breathing, such as rolling over.

FIGS. 8A and 8B are diagrams showing specific examples of a respiratory waveform and a body motion waveform separated from the waveform depicted in FIG. 7.

The respiratory waveform of a human being during “stable” sleep has periodicity. Therefore, when the periodicity of the respiratory waveform falls within a predetermined range, that is, when variations in cycle fall within a predetermined range, it can be the that “sleep” is generally stable.

During stable “sleep,” body motions other than breathing, such as rolling over, hardly occur. Therefore, when the amplitude of the body motion waveform falls within a predetermined range, it can be the that “sleep” is generally stable, whereas when it is equal to or greater than the predetermined range, it can be the that body motions occur and therefore “sleep” is not stable.

Accordingly, for a certain period, it can be determined whether the subject is in stable “sleep” based on the periodicity of the respiratory waveform in that period and the magnitude of body motions other than breathing. Although the determination preferably is made using both of the respiratory waveform and the body motion waveform in this example, only at least one of these waveforms may be used, for example.

As shown in FIG. 6, sleep state measurement unit 402 includes a determination unit 4021 and a correction unit 4022.

Determination unit 4021 separates the waveform based on the input sensor signal shown in FIG. 7 into the respiratory waveform and the body motion waveform shown in FIG. 8A and FIG. 8B. Based on those waveforms, it is determined whether the subject is in stable “sleep” for each predetermined unit period (periods t1, t2, t3, t4, t5 in FIG. 7). The unit period here is, for example, 30 seconds, 1 minute or so. That is, when cycle variations of the respiratory waveform in unit period t1 are smaller than a preset threshold value, it is determined that the respiratory waveform exhibits periodicity in unit period t1. It is also determined whether the amplitude of the body motion waveform in unit period t1 is greater or smaller than a preset threshold value.

When the respiratory waveform in unit period t1 has periodicity and the amplitude of the body motion waveform is smaller than the threshold value, determination unit 4021 determines that the sleep state of the subject in unit period t1 is “sleep” (S). On the other hand, when the respiratory waveform in unit period t1 does not have periodicity and the amplitude of the body motion waveform is greater than the threshold value, determination unit 4021 determines that the sleep state of the subject in unit period t1 is “wake” (W). When only one of the conditions is satisfied, that is, when the respiratory waveform in unit period t1 has periodicity or when the amplitude of the body motion waveform is smaller than the threshold value, it may be determined that the sleep state is “wake.”

Determination unit 4021 may determine whether the subject is present within the reach of radio waves output from body motion sensor 31.

In this determination, for example, after the waveform based on the sensor signal is separated into a respiratory waveform and a body motion waveform as described above, if the amplitude of either the respiratory waveform or the body motion waveform is smaller than a particular value for a particular period (for example, 30 seconds), determination unit 4021 determines that the subject is not present within the reach. Otherwise, determination unit 4021 determines that the subject is present within the reach. Determination unit 4021 determines on a state (E) when determining that the subject is present and determines on a state (A) when determining that the subject is not present (out of bed).

FIG. 9 is a diagram showing a specific example of determination results in determination unit 4021. As shown in FIG. 9, determination unit 4021 determines whether a state of sleep is “sleep” or “wake” for each unit period of the waveform based on the input sensor signal.

After determining a state of sleep for each unit period as described above, sleep state measurement unit 402 stores the result into memory 42.

Read unit 406 reads out the stored result of determination for each unit period and transmits the read result to communication control unit 408.

Communication control unit 408 allows communication unit 50 to transmit the determination result to server 500.

Server 500 thus acquires the determination result of a sleep state for each unit period.

Referring to FIG. 10, server 500 includes a control unit 540 that is programmed and configured to completely control operation of server 500. Server 500 further includes an operation unit 510, a display unit 520, and a communication unit 550, all of which are connected to control unit 40.

Server 500 may be implemented, for example, by a general personal computer.

Operation unit 510 may be implemented by an operating device such as a keyboard and a mouse. Operation unit 510 outputs an operation signal produced by an external operation to control unit 540.

Control unit 540 includes a CPU 541 configured to perform the complete control and a memory 542 configured to store a computer program executed in CPU 541.

Control unit 540 executes processing such as determination of a sleep level described later, by CPU 541 executing the program stored in memory 542 and performing operations using the input operation signal and sensor signal.

Memory 542 may be provided in or fixed to server 500 or may be implemented by a detachable storage medium. Examples of the storage medium include a CD-ROM, a DVD-ROM, a USB memory, a memory card, an FD, a hard disk, a magnetic tape, a cassette tape, an MO, an MD, an IC card (excluding a memory card), an optical card, a mask ROM, an EPROM, an EEPROM, and any other media storing programs in a nonvolatile manner.

Communication unit 550 preferably is implemented, for example, by a LAN card. Server 500 communicates with body motion detection device 100 and user terminal 200 through communication unit 550.

FIG. 11 is a diagram showing an example of a functional configuration of server 500.

In the present preferred embodiment, in body motion detection device 100, sleep state measurement unit 402 detects a state of sleep for each unit period. The detection result is then transmitted to server 500 by a data transfer unit 51. Data transfer unit 51 includes read unit 406, communication control unit 408, and communication unit 50.

Server 500 includes, as its functions, a sleep state storage unit 501, a sleep pattern index calculation unit 502, a sleep pattern index database operation unit 503, a sleep pattern index database 504, and a sleep pattern determination unit 505.

Sleep pattern index calculation unit 502, sleep pattern index database operation unit 503, and sleep pattern determination unit 505 are implemented by, for example, CPU 541 executing a computer program. At least a portion of these units may be implemented by a dedicated hardware component, for example. Sleep state storage unit 501 and sleep pattern index database 504 are preferably implemented by memory 542.

Sleep state storage unit 501 stores, for example, the detection result of a sleep state that is received from body motion detection device 100. Sleep pattern index calculation unit 502 performs processing for a result for each day of the received detection result (see FIG. 15 described later). Sleep pattern index database operation unit 503 stores the processing result for each day into sleep pattern index database 504. Sleep pattern determination unit 505 makes a determination as to a sleep pattern of the subject based on the detection result for two weeks stored in sleep pattern index database 504 and the processing result thereof (see FIG. 17 described later), for example.

In the present preferred embodiment, in server 500, data of a sleep state is processed for each day, and data of a plurality of days (for a certain period) is also processed. Although in the present preferred embodiment “a plurality of days” is “two weeks” by way of example, the length of “a plurality of days” is not limited thereto. The length may be longer or shorter than it. In the present description, “a plurality of days” in which measurement is performed is also expressed as “a certain period.”

In the present preferred embodiment, measurement related to human sleep is performed. Therefore, a day expressed as “each day” may not correspond to the day to which the time at which measurement is performed belongs. For example, in a case where the subject has a sleep from 11 p.m. on January 1 to 6 a.m. on January 2 as a sleep of Jan. 1, 2011, the measurement result is considered as the result of January 1 even though the latter half is included in January 2. In the present preferred embodiment, measurement results of two weeks are used as measurement results of a plurality of days, as described above. Strictly speaking, in order to obtain measurement results for two weeks from January 1, measurement may be performed from the night of January 1 to the morning of January 15, two weeks later, that is, for a period of two weeks plus one day.

The determination result by sleep pattern determination unit 505 is transmitted to user terminal 200, for example, in response to a request from user terminal 200. The determination result is displayed on user terminal 200.

As described above, server 500 receives the determination result of a sleep state for each unit period from body motion detection device 100. Control unit 540 then decides a sleep level based on the determination result of the sleep state.

Before deciding a sleep level, control unit 540 corrects the determination result in a unit period in accordance with the determination result of the adjacent unit period. Such correction is made considering that a reception signal of body motion detection device 100 weakens for a certain period due to rolling over, although the subject is in bed, and the determination result for each unit period becomes “unknown.” Such correction may be made considering that in a case where the subject is actually out of bed for a certain period and goes outside of the measurement range in a series of awake states, “wake” and “unknown” are mixed in a determination result in a unit period.

FIG. 12A to FIG. 12C are diagrams illustrating correction of a determination result of a sleep state and decision of a sleep level in server 500.

FIG. 12A shows a determination result of a sleep state that is transmitted from body motion detection device 100.

Referring to FIG. 12A and FIG. 12B, control unit 540 divides the data received from body motion detection device 100 into blocks for each unit period in which the determination results are the same in succession.

If the determination in a unit period that makes a block shows a certain state, and if the adjacent blocks before and after that block are in a particular same state, correction is made such that the determination in that block is supposed to be the determination result in the adjacent blocks. Correction may not be made depending on the number of such blocks and the state of adjacent blocks.

In server 500, a sleep level is then determined based on the determination results in each unit period for a predetermined period in which the unit periods are in succession.

In the present preferred embodiment, a predetermined period is, for example, about five minutes or ten minutes.

Here, a sleep level is defined by each state of “sleep,” “wake,” and “absent” the stability of breathing, presence/absence of body motion in each state, and continuity.

The following Levels 1 to 5 are shown as a specific example.

Level 1: “sleep” without body motions and with stable breathing.

Level 2: “sleep” with isolated body motion.

Level 3: “sleep” with isolated “wake.”

Level 4: “wake” with continuous body motion.

Level 5: “absent” in which the subject is outside of the measurement range for a certain period or longer.

FIG. 12C is a diagram showing a specific example of determination results of the sleep level for each predetermined period.

In FIG. 12C, T1, T2, T3 are shown as an example of the predetermined period.

CPU 541 makes a determination as to the tendency of sleep of the subject using the time information including the go-to-bed time and the wake-up time, the determination result for each unit period, or a sleep level for each predetermined period as described above.

In the present preferred embodiment, server 500 makes a determination as to a plurality of patterns of sleep of the subject, based on the time information including the go-to-bed time and the wake-up time, the determination result for each unit period, or the determined sleep level as described above. FIG. 13 is a diagram showing an example of the names of patterns determined in server 500 and the definition of criteria.

Referring to FIG. 13, the patterns determined in server 500 include six kinds, namely, “having difficulty falling asleep,” “awakening too early,” “awakening in the middle of night,” “shortage of sleep,” “sleep rhythm disturbance,” and “uninterrupted sleep.”

Among those, for “having difficulty falling asleep” “awakening too early,” and “awakening in middle of night,” a temporary determination is made for each day, and a final determination for a plurality of days is made based on the determination results for each day.

The determination criterion for each day for “having difficulty falling asleep” is that “the ‘sleep without body motions’ determination does not exist in 40 minutes after the start of measurement.” Specifically, CPU 541 determines whether a condition that “a determination result of Level 1 does not exist in 40 minutes after the start of measurement (the start of a sleep period described later)” occurs. If such a condition occurs, it is determined that the subject on that day fits in this pattern. If such a condition does not occur, it is determined that the subject does not fit in this pattern.

The determination criterion for “having difficulty falling asleep” for a plurality of days is whether there are seven or more days that fit in the pattern of “having difficulty falling asleep” in a determination for each day. If there are seven or more days that fit in this pattern, CPU 541 determines, as a final determination result, that the subject fits in the pattern of “having difficulty falling asleep.” If such a condition does not occur, it is determined that the subject does not fit in this pattern. As described above, in the present preferred embodiment, two weeks are set as “a plurality of days,” by way of example. For this pattern, however, the final determination result can be obtained for seven days after the start of measurement, at the earliest.

The determination criterion for each day of “awakening too early” is that “the total waking hours in 60 minutes before the end of measurement is 30 minutes or longer.” Specifically, CPU 541 determines whether a condition that “the total time in which a determination in a unit time is other than ‘sleep” in 60 minutes before the end of measurement (the end of a sleep period described later) is 30 minutes or longer” occurs. If such a condition occurs, it is determined that the subject on that day fits in this pattern. On the other hand, if such a condition does not occur, it is determined that the subject does not fit in this pattern.

The determination criterion for a plurality of days of “awakening too early” is whether there are seven or more days in which a threshold value for the above-noted total waking hours is exceeded in a determination for each day. If there are seven or more days that fit in this pattern, CPU 541 determines, as a final determination result, that the subject fits in the pattern of “awakening too early.” On the other hand, if such a condition does not occur, it is determined that the subject does not fit in this pattern. As described above, in the present preferred embodiment, two weeks are set as “a plurality of days,” by way of example. However, as for this pattern, a final determination result can be obtained for seven days after the start of measurement, at the earliest.

The determination criterion for each day of “awakening in the middle of night” is that “middle-of-the-night awakening occurs three or more times.” Specifically, CPU 541 determines whether a condition that “there are three or more intervals in which the above-noted Level 4 occurs during a sleep period as described later” occurs. If such a condition occurs, it is determined that the subject on that day fits in this pattern. On the other hand, if such a condition does not occur, it is determined that the subject does not fit in this pattern. Here, an “interval” occurs when a level other than Level 4 goes to Level 4 and returns to a level other than Level 4. In the present preferred embodiment, occurrence of such an interval corresponds to occurrence of “middle-of-the-night awakening.” In a determination of this pattern, CPU 541 determines whether such a manner of change that a level other than Level 4 goes to Level 4 and returns to a level other than Level 4 appears three or more times.

The determination criterion for a plurality of days of “awakening in the middle of night” is whether there are seven or more days in which the threshold value of the number of times of middle-of-the-night awakening as described above is exceeded in a determination for each day. If there are seven or more days that fit in this pattern, CPU 541 determines, as a final determination result, that the subject fits in the pattern of “awakening in the middle of night.” On the other hand, if such a condition does not occur, it is determined that the subject does not fit in this pattern. As described above, in the present preferred embodiment, two weeks are set as “a plurality of days” by way of example. However, for this pattern, a final determination result can be obtained for seven days after the start of measurement, at the earliest.

In server 500, a determination as to patterns called “shortage of sleep,” “sleep rhythm disturbance,” and “uninterrupted sleep” is further made.

The determination criterion for “shortage of sleep” is that “the average value of the total sleeping time for two weeks is less than six hours.” Specifically, CPU 541 determines whether a condition that “the average value for a plurality of days is calculated as to the length of a sleep period described later for each day, and the average value is less than six hours” occurs. If it is determined such a condition occurs, it is determined that the subject fits in the pattern of “shortage of sleep.” On the other hand, if such a condition does not occur, it is determined that the subject does not fit in this pattern.

The determination criterion for “sleep rhythm disturbance” is that “‘standard deviations of the go-to-bed time for two weeks are two hours or longer’ or ‘standard deviations of the wake-up time for two weeks are two hours or longer’.” Specifically, CPU 541 obtains standard deviations of the go-to-bed time (the start time of a sleep duration as described later) and standard deviations of the wake-up time (the end time of a sleep period as described later) for a plurality of days, and determines whether a condition that at least one of them is two hours or longer occurs. If it is determined that such a condition occurs, it is determined that the subject fits in the pattern of “sleep rhythm disturbance.” On the other hand, if such a condition does not occur, it is determined that the subject does not fit in this pattern.

Here, calculation of standard deviations will be described.

FIG. 14 is a diagram illustrating an example of a method of calculating standard deviations of the wake-up time.

FIG. 14 shows an equation (1) used to calculate standard deviations of the wake-up time for two weeks. In the present preferred embodiment, a standard deviation that is an index of sleep rhythm disturbance is calculated based on a deviation from the average value of the wake-up time for each day, and the average value. The calculation result of the standard deviation may be rounded off, as appropriate.

The standard deviations of the go-to-bed time are also calculated in the same manner as in the standard deviations of the wake-up time as described above.

The determination criterion for “uninterrupted sleep” is that “the average value for two weeks of the proportion of the ‘sleep without body motions’ determination in a one-day sleep is equal to or greater than a certain proportion.” Specifically, CPU 541 calculates the proportion of the total length of a predetermined period in which a determination of “Level 1” is made, to the length of a sleep period, for each day of a plurality of days, calculates the average value of the proportion for a plurality of days, and determines whether the average value is xx % (a preset value) or greater. Then, if it is determined that such a condition occurs, it is determined that the subject fits in the pattern of “uninterrupted sleep.” On the other hand, if such a condition does not occur, it is determined that the subject does not fit in this pattern.

FIG. 15 is a diagram illustrating contents of processing executed on the result for each day in the sleep evaluation system in the present preferred embodiment.

Referring to FIG. 15, in the sleep evaluation system, body motion detection device 100 detects a sleep state for each unit period at least during a sleep period for each day and transmits the detected sleep state to server 500.

In the present preferred embodiment, a sleep period refers to a period during which the subject is sleeping. A sleep period starts when the subject goes to bed, and ends when the subject wakes up. In body motion detection device 100, CPU 41 specifies the go-to-bed time, for example, at a timing when good night button 10C is operated, and specifies the wake-up time at a timing when good night button 10C is operated again. That is, in this system, the subject operates good night button 10C at a timing of “Now, sleep” after going to the bed, reading a book, etc. The subject then falls asleep. When waking up, the subject operates good night button 10C again.

In server 500, a determination as to the patterns “having difficulty falling asleep,” “awakening too early,” and “awakening in the middle of night” is made for each day, based on the determination result of a sleep state that is received from body motion detection device 100. The determination result, which is the determination result for each unit period as described above, includes “sleep” (S), “wake” (W), and “absent” (A) and further includes “unknown” (U) for a unit period that is determined not to fall into any of them.

In the sleep evaluation system, as shown in FIG. 15, data as to the magnitude of body motions (for example, the body motion waveform as shown in FIG. 8(B)) may be further transmitted from body motion detection device 100 to server 500. In this case, in server 500, a determination of a pattern for the subject is additionally performed using the body motion waveform.

In server 500, the indices for “shortage of sleep,” “sleep rhythm disturbance,” and “uninterrupted sleep” are extracted based on the determination result of a sleep state for a sleep period in each day for the subject.

The index for each day for “shortage of sleep” is the total sleeping time. The sleeping time refers to the length of a sleep period. CPU 541 calculates a sleeping time for each day by calculating the difference between the start time and the end time of a sleep period.

The index for each day for “sleep rhythm disturbance ” is a standard deviation of the go-to-bed time and a standard deviation of the wake-up time. The go-to-bed time is, for example, the start time of a sleep period. The wake-up time is, for example, the end time of a sleep period.

The index for each day for “uninterrupted sleep” is the ratio of the sum of the length of “a predetermined period” determined to be Level 1, to the length of a sleep period. For example, it is assumed that, as a determination result for a certain day, the length of a sleep period is six hours, the predetermined period is five minutes, there are 36 predetermined periods determined to be Level 1. In this case, the sum of the length of the predetermined period determined to be Level 1 is 5 minutes multiplied by 36, that is, 180 minutes (3 hours). Therefore, the ratio is 50%. The length of “a predetermined time” determined to be Level 1 corresponds to the length of the time in which the body motion detector detects that the body is not moved.

FIG. 16 is a diagram illustrating contents of processing for each day from the second day of measurement to the day previous to the last day (for example, the thirteenth day in the present preferred embodiment).

As described above, in the present preferred embodiment, it can be determined whether a result for each day fits in each of the patterns “having difficulty falling asleep,” “awakening too early,” and “awakening in the middle of night,” based on the determination result of a sleep state for each day. A determination to derive a final result is also possible for each pattern from the second day to the day previous to the final day. That is, from the second day to the day previous to the final day, it is determined whether the result fits in “the determination criterion for a plurality of days” described with reference to FIG. 13, based on the preceding determination results for each day.

FIG. 17 is a diagram illustrating contents of processing to determine a sleep tendency for a plurality of days (for a certain period) which are a period of time to provide a final determination result to the subject.

The final determination result for each pattern is executed, for example, on the final day of a measurement period (in the present preferred embodiment, the fourteenth day).

In server 500, a final determination as to the six kinds of patterns as described above is made based on the measurement results for a plurality of days.

Specifically, for each of the patterns “having difficulty falling asleep,” “awakening too early,” and “awakening in middle of night,” a final pattern determination is made based on “the determination results for each day” for a plurality of days.

For these patterns, a final determination may be made each day based on “the determination result for each day” until that day. If a determination is made in this manner, as described above, a final determination may be completed for four days, at the earliest, before expiration of a plurality of days.

For “shortage of sleep,” “sleep rhythm disturbance,” and “uninterrupted sleep,” a final pattern determination is made based on the index obtained from a detection result for each day.

FIG. 17 shows an example of the final determination result for each of the six kinds of patterns.

CPU 541 performs, as output processing, generation of data for displaying a result for determination, for example. The thus generated data is transmitted as a determination result to user terminal 200.

Preferably, CPU 541 outputs a determination result to an external device such as user terminal 200, based on a condition that a certain period described above (a plurality of days) is expired. That is, in the present preferred embodiment, for “having difficulty falling asleep,” “awakening too early,” and “awakening in the middle of night,” a final determination result for the pattern may be made before expiration of a certain period. However, if a final determination is presented to user terminal 200 based on a condition that a final determination result is finalized for some of the patterns, inconveniently, the timing for presentation varies among users. Therefore, it is preferable that server 500 outputs a determination result based on a condition that a final determination result is finalized for all the patterns, or based on a condition that a certain period is expired.

FIG. 18 is a diagram showing an example of a screen used to display the result of determination described above.

A screen 900 in FIG. 18 includes a field 901 to display presence/absence of self-consciousness of the subject for each pattern, a field 902 to display a determination result, a field 903 to display a numerical value for each pattern with a graph, and a field 904 to directly display a numerical value.

Field 901 includes a checkbox for each pattern. A tick mark may be displayed in the checkbox.

In FIG. 18, the pattern “having difficulty falling asleep” is shown by “difficulty falling asleep,” the pattern “awakening too early” is shown by “awaken early in the morning,” the pattern “awakening in the middle of night” is shown by “awakening in the middle of night,” the pattern “shortage of sleep” is shown by “shortage of sleep,” the pattern “sleep rhythm disturbance” is shown by “rhythm,” and the pattern “uninterrupted sleep” is shown by “uninterrupted sleep.”

CPU 541 of server 500 accepts input of information to report whether the subject is conscious of each pattern, together with a request for a determination result, from user terminal 200. CPU 541 allows a tick mark to be displayed in the corresponding checkbox for the pattern that the subject reports being conscious of, and does not allow such a tick mark to be displayed for the pattern that the subject reports not being conscious of.

In the present preferred embodiment, the detection result in body motion detection device 100 is displayed together with the content that the subject himself/herself has reported, such that screen 900 includes field 901. Accordingly, the measurement result preferably is provided to the subject together with his/her own impression on his/her sleep, so that the measurement result is provided such that the measurement result makes an impression on the subject. In a case where such a measurement result is provided to the doctor in charge of the subject, the doctor can develop therapeutic plans for the subject's sleep more appropriately.

Only information to display each checkbox may be transmitted from server 500 to user terminal 200. In this case, for example, in user terminal 200, a report as to whether the subject is conscious of each pattern is accepted, and whether to display a tick mark in each checkbox is decided.

Field 902 displays applicable/not applicable for each pattern. A smile face or a cry face preferably is displayed in a section in field 902 corresponding to each pattern. Specifically, in FIG. 18, displayed in a section in field 902 corresponding to “awaken early in the morning” is “smile face,” and displayed in a section in field 902 corresponding to “difficulty falling asleep” is “cry face.” In FIG. 18, for the patterns except “uninterrupted sleep,” a cry face is displayed if applicable, and a smile face is displayed if not applicable. This is because a preferable sleep is the one that is not applicable to these patterns. On the other hand, for “uninterrupted sleep,” a smile face is displayed if applicable to the pattern, and a cry face is displayed if not applicable. This is because it is preferable to be applicable to this pattern.

A display content in a section in field 904 corresponding to each pattern will be described.

In a section “difficulty falling asleep,” the average value of the time from the start of measurement until a determination result of Level 1 first appears, in determination results for each day, is shown.

In a section “awaken early in the morning,” the average value of the sum of the time determined to be other than “sleep” as a determination result for unit period in 60 minutes before the end of measurement in a determination result for each day is displayed.

In a section “awakening in the middle of night,” the average value for the number of times the aforementioned “interval” of middle-of-the-night awakening occurs, in a determination result for each day, is displayed.

In a section “lack of sleep,” the average value for the length of a sleep period, in a determination result for each day, is displayed.

In a section “rhythm,” the average value of deviations from the average value for the wake-up time illustrated in FIG. 14 (or deviations from the average value of the go-to-bed time), in a determination result for each day, is displayed.

In a section “uninterrupted sleep,” the average value of indices for each day as to “uninterrupted sleep” as explained with reference to FIG. 15 is displayed.

In the present preferred embodiment as described above, body motion detection device 100 detects body motions of the subject and transmits the detection result to server 500. In server 500, a determination as to six kinds of patterns of sleep is made. In this case, the sleep evaluation device is implemented by server 500. For example, the acquisition unit is configured with communication unit 550 of server 500.

Body motion detection device 100 may have the function of server 500, and body motion detection device 100 may make a determination as to such patterns per se. In this case, information to display a determination result as shown in FIG. 18 is transmitted directly from body motion detection device 100 to user terminal 200. In this case, the sleep evaluation device preferably is configured with body motion detection device 100.

The preferred embodiments disclosed here should be understood as being illustrative rather than being limitative in all respects. The scope of the present invention is determined or limited by the foregoing description but in the claims, and it is intended that all modifications within the meaning and range of equivalence to the claims are embraced herein.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

1-6. (canceled)

7. A sleep evaluation device comprising:
an acquisition unit configured to acquire a detection result as to movement of a body of a subject in bed;
a first determination unit configured to determine a sleep state of the subject for each day, based on the detection result; and
a second determination unit configured to determine a tendency of the sleep state of the subject, based on determination results for a plurality of days by the first determination unit; wherein
the second determination unit is configured to determine the tendency of the sleep state of the subject for a plurality of items;
the plurality of items include items having different periods required for determination; and
the second determination unit is configured to provide a notice of the tendency of the sleep state of the subject, based on a condition that a period required for determination for all of the plurality of items is expired after the first determination unit starts determination.

8. The sleep evaluation device according to claim 7, wherein the items for which the tendency is determined by the second determination unit include an item based on a proportion of a time during which the body is not moved in the detection result, to a sleep duration.

9. A non-transitory computer-readable medium including a computer program for performing, when the computer program runs on a computer, a method of evaluating sleep of a subject including the steps of:
acquiring a detection result for movement of a body of a subject in bed that is detected by a body motion detection unit;
determining a sleep state of the subject for each day, based on a detection result of the body motion detection unit; and
determining a tendency of the sleep state of the subject, based on determination results of the sleep state for a plurality of days; wherein
the step of determining the tendency of the sleep state of the subject determines the tendency of the sleep state of the subject for a plurality of items;
the plurality of items include items having different periods required for determination; and
the step of determining the tendency of the sleep state of the subject provides a notice of the tendency of the sleep state of the subject, based on a condition that a period required for determination for all of the plurality of items is expired after the determination of a sleep state of the subject for each day is started.
